(19) United States
(12) Patent Application Publication
(10) Pub. No.: US 2018/0279878 A1
(43) Pub. Date: Oct. 4, 2018

(54) MRI SYSTEM, MRI APPARATUS, AND MRI METHOD

(71) Applicant: CANON MEDICAL SYSTEMS CORPORATION, Otawara-Shi (JP)

(72) Inventors: Ei NOMURA, Tsukuba (JP); Hitoshi KANAZAWA, Utsunomiya (JP)

(73) Assignee: CANON MEDICAL SYSTEMS CORPORATION, Otawara-Shi (JP)

(21) Appl. No.: 15/943,843

(22) Filed: Apr. 3, 2018

(30) Foreign Application Priority Data
Apr. 4, 2017 (JP) 2017-074351
Mar. 27, 2018 (JP) 2018-060342

(51) Int. Cl. A61B 5/00; A61B 5/055; A61B 5/026; G01R 33/563

(52) U.S. Cl. CPC A61B 5/004; A61B 5/055; G01R 33/5635; A61B 5/0263; A61B 5/7285

(57) ABSTRACT

In one embodiment, an MRI apparatus is configured to be connected with a pressure device that externally pressures a blood vessel of an object, and includes a scanner configured to perform imaging on the object and processing circuitry. The processing circuitry controls the pressure device in such a manner that ischemia and reperfusion are caused with respect to the object, determines a start timing of the imaging on the basis of a state of pressurization caused by the pressure device, causes the scanner to perform the imaging on the object in accordance with the start timing, and generates an image by using data acquired in the imaging.

### CROSS-REFERENCE TO RELATED APPLICATIONS

This application is based upon and claims the benefit of priority from Japanese Patent Application No. 2017-074351, filed on Apr. 4, 2017 and Japanese Patent Application No. 2018-060342 filed on Mar. 27, 2018, the entire contents of each of which are incorporated herein by reference.

### FIELD

Embodiments described herein relate generally to a magnetic resonance imaging (MRI) apparatus, an MRI system, and an MRI method.

### BACKGROUND

An MRI apparatus is an imaging apparatus that magnetically excites nuclear spin of an object placed in a static magnetic field with a radio frequency (RF) pulse having the Larmor frequency and reconstructs an image on the basis of magnetic resonance (MR) signals emitted from the object due to the excitation.

Among imaging methods using an MRI apparatus, there is a known imaging method called non-contrast enhanced MRA (Magnetic Resonance Angiography) in which blood vessels and blood flow are imaged without using a contrast medium.

The non-contrast enhanced MRA includes imaging methods based on different imaging principles, such as a time-of-flight (TOF) method, a phase contrast (PC) method, a fresh blood imaging (FBI) method, and a time-spatial labelling inversion pulse (Time-SLIP) method.

When imaging is performed under any one of the above-described various types of non-contrast enhanced MRA in conventional technology, imaging capability of blood vessels may be deteriorated due to physiological factors as described below.

For instance, in the case of a patient with heart disease, the blood flow is decreased due to circulatory insufficiency and thus the influx effect of blood into each imaging plane is weakened, which may consequently deteriorate imaging capability of blood vessels.

Further, in the case of, e.g., a patient whose cardiac cycle is not stable due to chronic disease such as an arrhythmia, MR signals cannot be acquired at an intended imaging timing (e.g., systole and diastole) in the non-contrast enhanced MRA using electrocardiographic (ECG) synchronization, which may consequently deteriorate imaging capability of blood vessels.

### BRIEF DESCRIPTION OF THE DRAWINGS

In the accompanying drawings:

FIG. 1 is a block diagram illustrating overall configuration of an MRI apparatus according to the first and second embodiments;

FIG. 2 is a block diagram illustrating configuration related to RIC-combined imaging of an MRI system according to the first embodiment;

FIG. 3 is a schematic diagram illustrating a concept of an RIC mechanism;

FIG. 4 is a schematic diagram illustrating a concept of a blood-flow increasing effect of RIC effects;

FIG. 5 is a schematic diagram illustrating relationship between decrease in imaging capability due to physiological factors in various imaging methods included in the category of non-contrast enhanced MRA and improvement effect of imaging capability due to RIC effects;

FIG. 6 is a flowchart illustrating a detailed operation of the MRI system and the MRI apparatus of the first embodiment;

FIG. 7 is a schematic diagram illustrating a concept of a pulse-wave transit time;

FIG. 8 is a graph illustrating relationship between a pulse-wave transit time and blood flow;

FIG. 9 is a timing chart illustrating the operation concept of the first embodiment;

FIG. 10 is a flowchart illustrating an operation of the second embodiment;

FIG. 11 is a schematic diagram illustrating a concept of images generated in the second embodiment;

FIG. 12 is a schematic diagram illustrating an axial cross-section of the brain in which an acute cerebral infarction occurs;

FIG. 13 is a schematic diagram illustrating changes in respective regions of an infarct core and an ischemic penumbra depending on elapsed time since onset;

FIG. 14 is a schematic diagram illustrating a processing concept of the third embodiment;

FIG. 15 is a block diagram illustrating configuration related to RIC-combined imaging of an MRI system according to the third embodiment;

FIG. 16 is a flowchart illustrating an operation of the MRI system and the MRI apparatus of the third embodiment;

FIG. 17 is a schematic diagram illustrating a pulse sequence of IVIM imaging;

FIG. 18 is a graph illustrating a concept of signal intensity ratio to b-value in a specific one voxel of an IVIM image;

FIG. 19 is a graph schematically illustrating change in characteristics of signal intensity ratio to b-value before and after RIC; and

FIG. 20 is a schematic diagram illustrating a concept of distinguishing between an ischemic penumbra and an infarct core by using difference Δf in reperfusion fraction before and after execution of RIC.

### DETAILED DESCRIPTION

Hereinafter, respective embodiments of an MRI apparatus, an MRI system, and an MRI method will be described with reference to the accompanying drawings.

In one embodiment, an MRI apparatus is configured to be connected with a pressure device that externally pressures a blood vessel of an object. The MRI apparatus includes a scanner configured to perform imaging on the object and processing circuitry. The processing circuitry controls the pressure device in such a manner that ischemia and reperfusion are caused with respect to the object, determines a start timing of the imaging on the basis of a state of pressurization caused by the pressure device, causes the scanner to perform the imaging on the object in accordance with the start timing, and generates an image by using data acquired in the imaging.

### First Embodiment

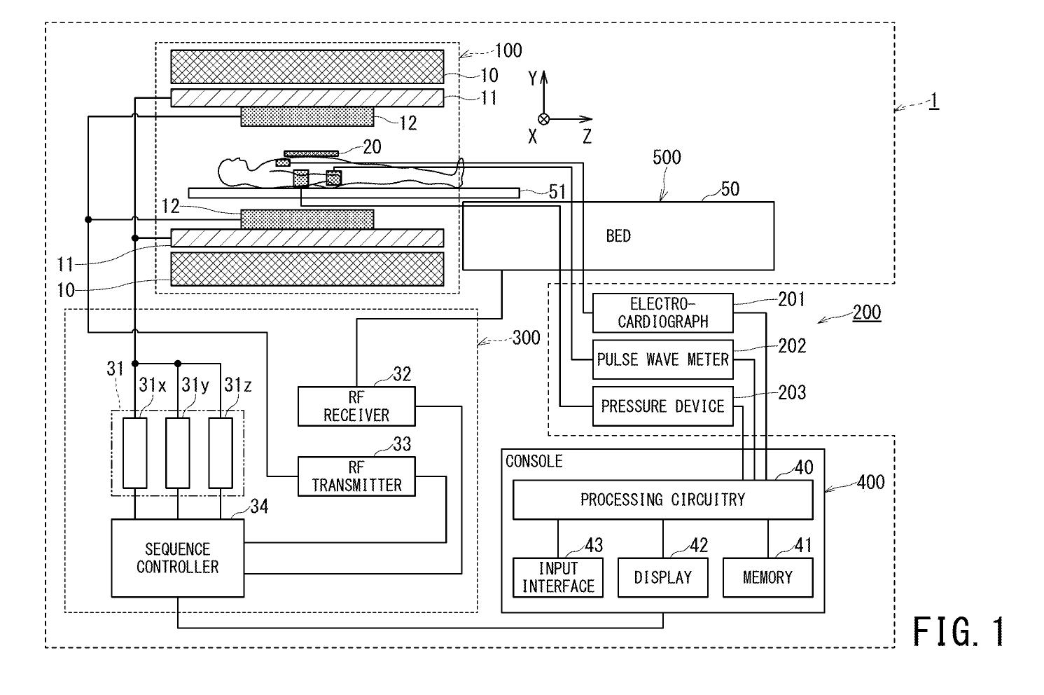

FIG. 1 is a block diagram illustrating overall configuration of an MRI apparatus 1 and an MRI system 200 according to the first embodiment. The MRI apparatus 1 of first embodiment includes at least a gantry 100, a control cabinet 300, a console 400, and a bed 500.

In addition to the configuration of the MRI apparatus 1, the MRI system 200 of the first embodiment also includes an electrocardiograph 201, a pulse wave meter 202, and a pressure device 203. In the following, the configuration of the MRI apparatus 1 will be described first.

The gantry 100 includes, e.g., a static magnetic field magnet 10, a gradient coil 11, and a whole body (WB) coil 12, and these components are housed in a cylindrical housing. The bed 500 includes a bed body 50 and a table 51.

Additionally, the MRI apparatus 1 further includes an array coil 20 to be placed onto an object.

The control cabinet 300 includes three gradient coil power supplies 31 (31x for an X-axis, 31y for a Y-axis, and 31z for a Z-axis), an RF receiver 32, an RF transmitter 33, and a sequence controller 34.

The static magnetic field magnet 10 of the gantry 100 is substantially in the form of a cylinder, and generates a static magnetic field inside a bore, which is an imaging region of an object (i.e., a patient). The bore is a space inside the cylindrical structure of the static magnetic field magnet 10. The static magnetic field magnet 10 includes a superconducting coil inside, and the superconducting coil is cooled down to an extremely low temperature by liquid helium. The static magnetic field magnet 10 generates a static magnetic field by supplying the superconducting coil with an electric current provided from a non-illustrated static magnetic field power supply in an excitation mode. Afterward, the static magnetic field magnet 10 shifts to a permanent current mode, and the static magnetic field supply is separated. Once it enters the permanent current mode, the static magnetic field magnet 10 continues to generate a strong static magnetic field for a long time, e.g., over one year. Note that the static magnetic field magnet 10 may be configured as a permanent magnet.

The gradient coil 11 is also substantially in the form of a cylinder and is fixed to the inside of the static magnetic field magnet 10. This gradient coil 11 applies gradient magnetic fields (e.g., gradient pulses) to an object in the respective directions of the X-axis, the Y-axis, and the Z-axis, by using electric currents supplied from the gradient coil power supplies 31x, 31y, and 31z.

The bed body 50 of the bed 500 can move the table 51 in the upward and downward directions, and moves the table 51 with an object loaded thereon to a predetermined height before imaging. Afterward, when the object is imaged, the bed body 50 moves the table 51 in the horizontal direction so as to move the object to inside of the bore.

The WB body coil 12 is shaped substantially in the form of a cylinder so as to surround the object, and is fixed to the inside of the gradient coil 11. The WB coil 12 applies RF pulses transmitted from the RF transmitter 33 to the object, and receives MR signals emitted from the object due to excitation of hydrogen nuclei.

The array coil 20 is an RF coil and receives MR signals emitted from the object at positions adjacent to the object. The array coil 20 is, for instance, configured of plural coil elements. Although there are various types for the array coil 20 such as a head coil, a chest coil, a spine coil, a lower limb coil, and a whole-body type coil according to an anatomical imaging part of the object, the array coil 20 for the chest part is illustrated in FIG. 1.

The RF transmitter 33 transmits RF pulses to the WB coil 12 on the basis of commands inputted from the sequence controller 34.

The RF receiver 32 receives MR signals received by the WB coil 12 and/or the array coil 20, and transmits raw data obtained by digitizing the received MR signals to the sequence controller 34.

The sequence controller 34 performs a scan of the object by driving the gradient coil power supplies 31, the RF transmitter 33, and the RF receiver 32, under the control of the console 400. When the sequence controller 34 receives raw data from the RF receiver 32 by performing a scan, the sequence controller 34 transmits the received raw data to the console 400.

The sequence controller 34 includes non-illustrated processing circuitry. This processing circuitry is configured as, e.g., a processor for executing predetermined programs or configured as hardware such as a field programmable gate array (FPGA) or an application specific integrated circuit (ASIC).

Of the components of the MRI apparatus 1 shown in FIG. 1, all the components except the console 40 constitute an imaging unit 600 that is referred to as a scanner 600. In other words, the control cabinet 300, the gantry 100, and the bed 500 constitute the scanner 600.

The console 400 is configured as a computer that includes processing circuitry 40, a memory 41, a display 42, and an input interface 43.

The memory 41 is a recording medium including a read-only memory (ROM) and a random access memory (RAM) in addition to an external memory device such as a hard disk drive (HDD) and an optical disc device. The memory 41 stores various programs executed by a processor of the processing circuitry 40 as well as various types of data and information.

The input interface 43 includes various devices for an operator to input various types of information and data, and is configured of, e.g., a mouse, a keyboard, a trackball, and/or a touch panel.

The display 42 is a display device such as a liquid crystal display panel, a plasma display panel, and an organic EL panel.

The processing circuitry 40 is, e.g., a circuit equipped with a central processing unit (CPU) and/or a special-purpose or general-purpose processor. The processor implements various functions described below by executing programs stored in the memory 41. The processing circuitry 40 may be configured of hardware such as an FPGA and an ASIC. The various functions described below can also be implemented by such hardware. Additionally, the processing circuitry 40 can implement the various functions by combining hardware processing and software processing based on its processor and programs.

The console 400 controls the entirety of the MRI apparatus 1 by controlling the above-described components. Specifically, the console 400 receives commands and various types of information such as imaging conditions inputted via, e.g., a mouse and a keyboard (of the input interface 42) operated by an operator such as an MRI technologist. Further, the processing circuitry 40 causes the sequence controller 34 to perform a scan on the basis of the inputted imaging conditions, and then reconstructs images on the basis of raw data transmitted from the sequence controller 34. The reconstructed images are displayed on the display 42 and stored in the memory 41.

As described above, the MRI system 200 includes the electrocardiograph 201, the pulse wave meter 202, and the pressure device 203 in addition to the configuration of the MRI apparatus 1.

The electrocardiograph 201 generates electrocardiogram (ECG) signals including an R-wave on the basis of signals outputted from plural electrodes that are attached to the body surface of the object's chest.

The pulse wave meter 202 is a device for measuring a pulse wave that is change in blood pressure or volume in the peripheral vasculature in association with heartbeat. The pulse wave is usually measured at the fingertip of the hand, and the pulse wave meter 202 of the present embodiment also measures the pulse wave on the basis of the signals from a pulse wave probe attached to the fingertip of the object's hand. The pulse wave meter 202 may be a measuring instrument called, e.g., a pulse oximeter, and measures arterial blood fluctuation (i.e., pulse wave) by optically measuring the percutaneous oxygen saturation (SpO₂) of the fingertip of the hand.

The pressure device 203 includes, e.g., an inflatable cuff 204, a pump, and a tube. The inflatable cuff 204 is a fastener corresponding to a cuff (arm band) of a sphygmomanometer (i.e., a blood pressure meter). For instance, by sending air from the pump through the tube to the inflatable cuff 204 wound around the arm, the inflatable cuff 204 is inflated so as to tighten blood vessels of the arm and the pressure corresponding to the internal pressure of the cuff is applied to the blood vessels. The pressure applied to the blood vessels can be monitored as the internal pressure of the cuff, and the unit of this pressure is usually expressed in mmHg.

As the applied pressure of the inflatable cuff 204 wound around the arm is increased, the blood vessels get crushed and the blood supply to the peripheral tissues immediately becomes insufficient. This state is called an ischemic state or simply called ischemia. After falling into the ischemic state, as the inflatable cuff 204 is loosened (i.e., as the applied pressure of the inflatable cuff 204 is lowered), the blood begins to flow. This phenomenon is called reperfusion.

As described below, it is known that ischemia tolerance reaction (i.e., endogenous defense function against ischemia), which a living body originally has, occurs by repeating the cycle of applying pressure once or plural times to a site distant from the heart (e.g., arm) with the inflatable cuff 204 to cause the ischemic state and then relaxing the inflatable cuff 204 to cause reperfusion. Note that the above-described action of repeating the ischemia and reperfusion plural times is called “remote ischemic conditioning” (hereinafter, referred to simply as “RIC”). The RIC is a method of inducing endogenous tolerance (cytoprotective phenomena) by artificial mild ischemic load. The RIC is expected to be applied to therapy such as reduction of cell damage in ischemic diseases such as cerebral infarction or myocardial infarction.

The MRI system 200, the MRI apparatus 1, and the MRI method of the present embodiment enhance the imaging capability of non-contrast enhanced MRA or contrast-enhanced MRA by using physiological effects that are caused by RIC (hereinafter, referred to as “RIC effects”). Further, imaging with the use of the RIC effects is hereinafter referred to as “RIC-combined imaging”. As described below, a blood-flow increasing effect or an antiarrhythmic effect are known as one of the RIC effects. Although a description will be mainly given of embodiments of the MRI system 200, the MRI apparatus 1, and the MRI method using non-contrast enhanced MRA in the following, embodiments of the present invention are not limited to non-contrast enhanced MRA but can also be applied to embodiments of contrast-enhanced MRA.

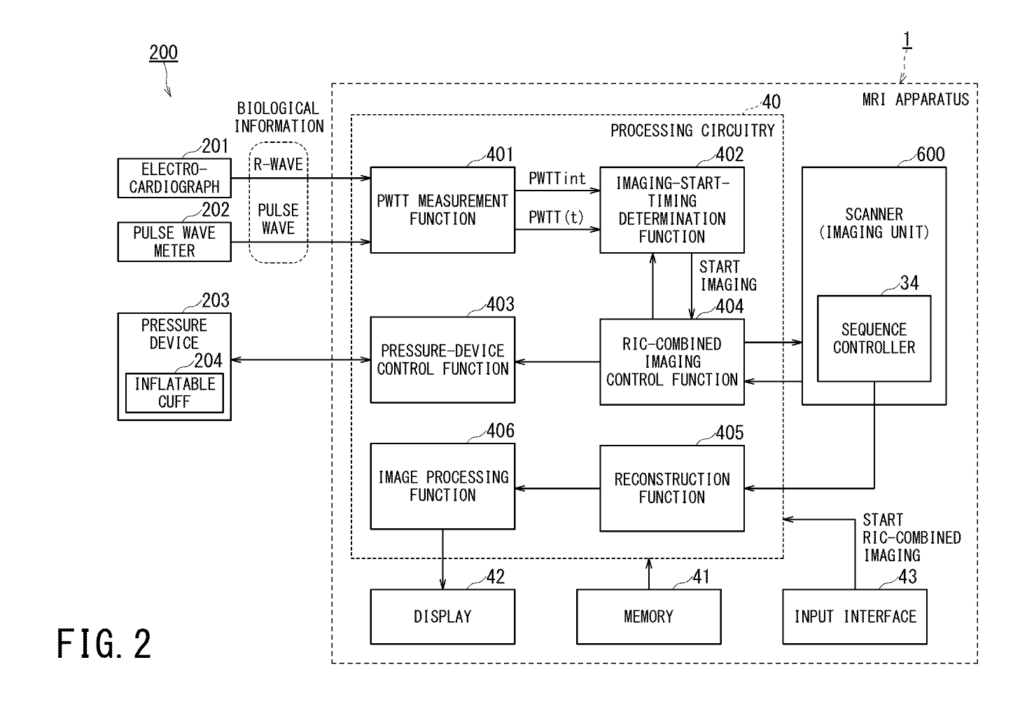

FIG. 2 is a functional block diagram of the MRI system 200 (and the MRI apparatus 1) of the present embodiment for performing the RIC-combined imaging by using the RIC effects.

As described above, the MRI system 200 includes the MRI apparatus 1, the electrocardiograph 201 connected to the MRI apparatus 1, the pulse wave meter 202 connected to the MRI apparatus 1, and the pressure device 203 equipped with the inflatable cuff 204.

As shown in FIG. 2, the processing circuitry 40 of the MRI apparatus 1 implements respective functions including a pulse-wave transit time (PWTT) measurement function 401, an imaging-start-timing determination function 402, a pressure-device control function 403, an RIC-combined imaging control function 404, a reconstruction function 405, and an image processing function 406 by, e.g., causing its processor to execute predetermined programs.

When the RIC-combined imaging control function 404 receives an imaging start command from a user through an operation on the input interface 43, the RIC-combined imaging control function 404 firstly issues a command for the pressure-device control function 403 to control the pressure device 403, instead of immediately starting imaging.

Upon receiving this command, the pressure-device control function 403 starts controlling the pressure device 203. Specifically, the pressure device 203 is controlled so as to repeat the cycle of bringing the blood vessels into ischemia by applying pressure with the inflatable cuff 204 and then reperfusing the blood by loosening the inflatable cuff 204.

Meanwhile, the PWTT measurement function 401 and the imaging-start-timing determination function 402 determine an imaging start timing on the basis of the biological information of the object that is obtained according to the pressurization of the blood vessels to be performed by the pressure device 203.

Specifically, the PWTT measurement function 401 measures a pulse wave transit time (PWTT) as the biological information of the object, on the basis of ECG signals outputted from the electrocardiograph 201 and pulse wave signals outputted from the pulse wave meter 202.

The imaging-start-timing determination function 402 determines the start timing of the RIC-combined imaging by using the ratio between the initial pulse wave transit time PWTTinit measured before the pressurization (or measured before start of the ischemia-and-reperfusion cycle) and the pulse wave transit time PWTT measured after start of the pressurization (or measured after start of the ischemia-and-reperfusion cycle).

When the start timing of the RIC-combined imaging is determined by the imaging-start-timing determination function 402, the RIC-combined imaging control function 404 issues a command for the sequence controller 34 of the scanner 600 to actually start imaging.

More detailed functions of the PWTT measurement function 401, the imaging-start-timing determination function 402, the pressure-device control function 403, and the RIC-combined imaging control function 404 will be described below.

Imaging methods to which the present embodiment is applied are mainly non-contrast enhanced MRA. When imaging is started, MR signals from the object is acquired and then the reconstruction function 405 performs reconstruction processing such as inverse Fourier transform on the MR signals so as to generate a blood vessel image as a real space image.

The reconstructed real space image is subjected to predetermined image processing such as maximum intensity projection (MIP) processing or predetermined rendering processing by the image processing function 406. The blood vessel image subjected to the above-described processing is outputted to the display 42 and displayed.

(Mechanism of RIC and RIC Effects)

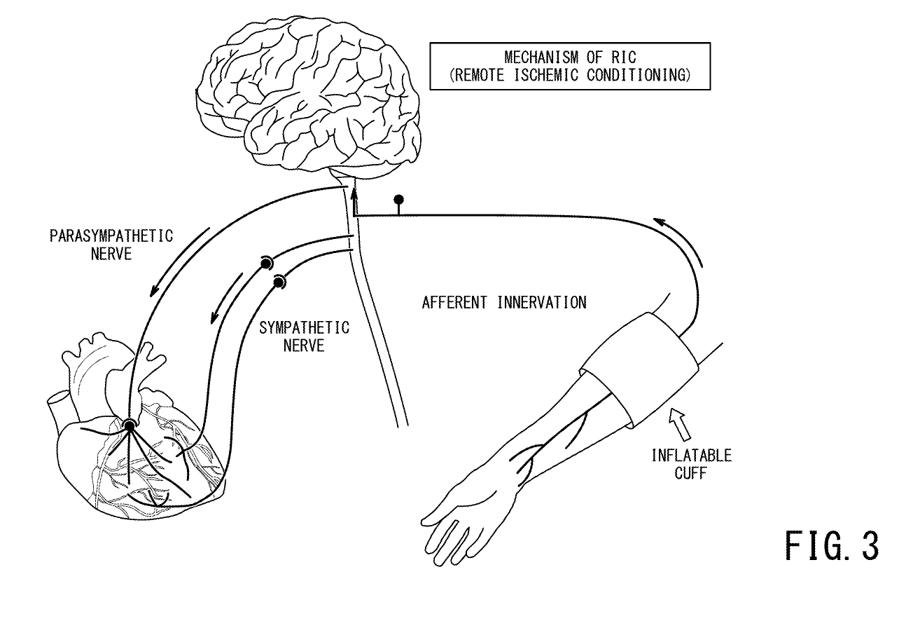

FIG. 3 is a schematic diagram illustrating the concept of the RIC mechanism. As described above, the mechanism of RIC is outlined below. First, ischemia and reperfusion are repetitively caused by repeating tightening and relaxation of the inflatable cuff 204 attached to a site remote from the heart (e.g., the upper arm). By repeating ischemia and reperfusion, neural information called afferent innervation is transmitted to the brain. Then, stimulation is given from the brain to the heart via sympathetic nerves and parasympathetic nerves. Biological response based on this RIC mechanism is also called as remote preconditioning reflex. The remote preconditioning reflex can be regarded as a biological reaction for activating the activity of the heart and supplementing deficient substances due to ischemia such as oxygen.

It is known that at least the following two RIC effects are obtained when the stimulus is given from the brain to the heart by the above-described mechanism.

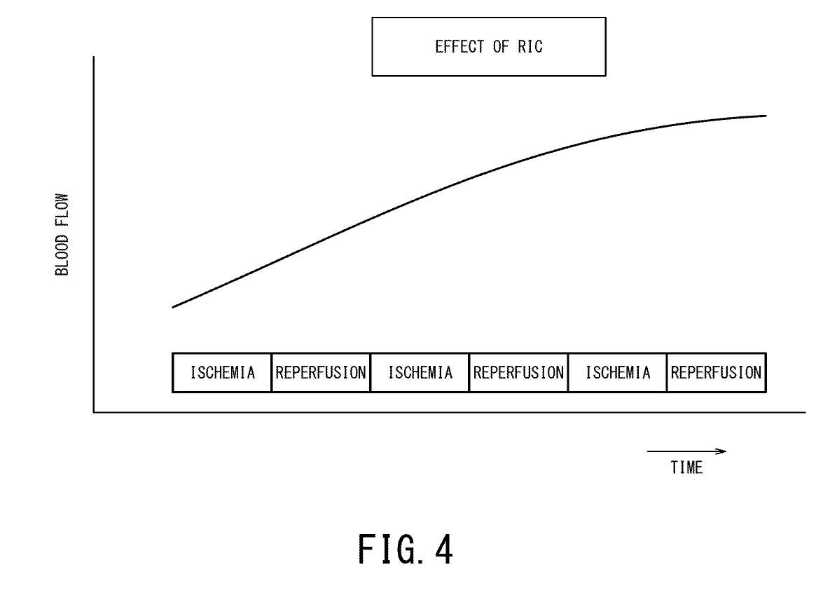

The first RIC effect is an effect of increasing blood flow. FIG. 4 is a schematic diagram illustrating the concept of the blood-flow increasing effect among the RIC effects. It is known that repetition of the ischemia-and-reperfusion cycle by controlling the pressure device 203 results in a gradual increase in the average blood flow as shown in FIG. 4, even though there is a small fluctuation in blood flow within the period of ischemia and/or within the period of reperfusion. This biological phenomenon caused by RIC is the blood-flow increasing effect.

The second RIC effect is an antiarrhythmic effect in which arrhythmia is suppressed and the cardiac cycle is stabilized. It is known that the antiarrhythmic effect is obtained by repeating the ischemia-and-reperfusion cycle for an object whose cardiac cycle is unstable with the use of the inflatable cuff 204 attached to, e.g., the upper arm in a similar manner as described above.

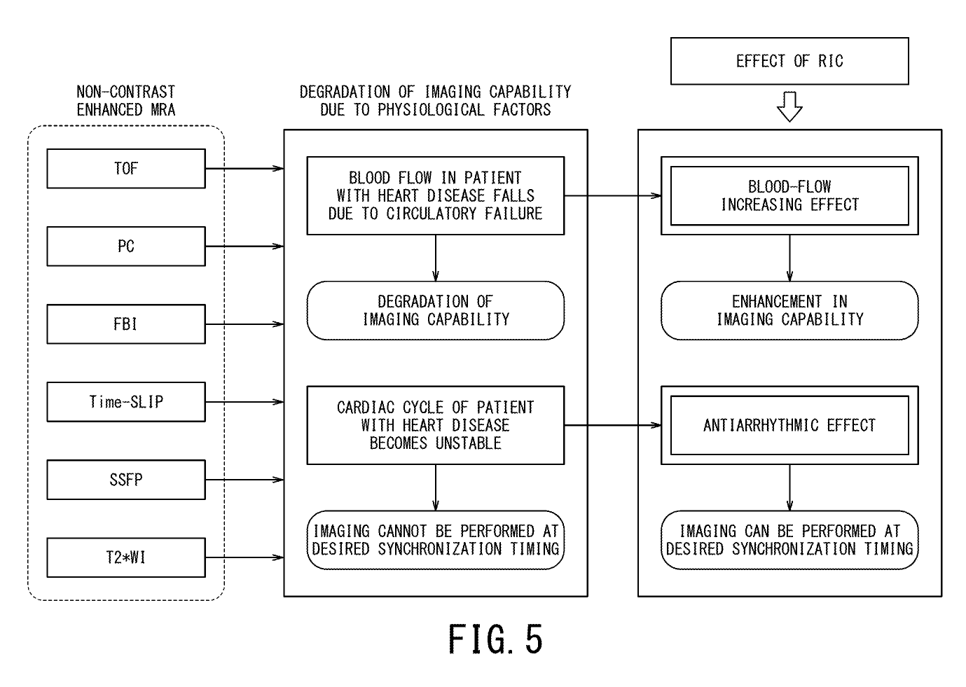

In many imaging methods of non-contrast enhanced MRA, it is known that imaging capability is reduced due to physiological factors. FIG. 5 is a schematic diagram illustrating relationship between (a) degradation of imaging capability due to physiological factors in various imaging methods included in the category of non-contrast enhanced MRA and (b) effect of improving the imaging capability by the above-described RIC effects.

The column on the most left side of FIG. 5 lists various imaging methods of non-contrast enhanced MRA. Among these imaging methods, the TOF (Time-of-flight) method is an imaging method of acquiring a blood vessel image by using an inflow effect of fresh blood spin into an imaging plane. The PC (Phase Contrast) method is an imaging method of acquiring a blood vessel image by using phase difference in blood spin caused by the blood flow.

The FBI (Fresh Blood Imaging) method is an imaging method of acquiring a blood vessel image by using difference in contrast or blood flow velocity between blood, water and tissues. According to the FBI method, it is possible to generate a satisfactory blood vessel image, in which an artery and a vein are clearly separated, by, e.g., performing difference processing on respective two images acquired in systole and diastole.

In addition, the Time-SLIP (Time-Spatial Labeling Inversion Pulse) method is an imaging method in which blood vessel morphology and blood dynamics can be imaged from labeled blood protons. According to the Time-SLIP method, for instance, it is possible to obtain a blood vessel image with a satisfactorily suppressed background by performing difference processing between an image acquired by applying a region selective inversion pulse as a labeling pulse and an image acquired without application of the labeling pulse.

Further, the SSFP (Steady-state Free Precession) method has a gradient magnetic field waveform of flow-rephase type and has T2/T1-weighted contrast. Thus, blood with a longer T2 causes a high signal in the SSFP method, and the SSFP method is suitable for depicting flow. Moreover, the T2*WI method is an imaging method of using difference in magnetic susceptibility depending on blood oxygen level.

Imaging capability of each of these various imaging methods belonging to non-contrast enhanced MRA depends on the blood flow in blood vessels. However, patients with heart disease have a physiological factor that blood flow decreases due to circulatory failure, which deteriorates the imaging capability of these various imaging methods.

As described above, it is possible to obtain an effect of increasing blood flow by performing RIC. In other words, by applying ischemia and reperfusion repeatedly to a patient with heart disease, the decreased blood flow can be increased and the imaging capability can be improved.

In these various imaging methods of non-contrast enhanced MRA, ECG-gated (i.e., electrocardiographic synchronization) imaging is often performed. The ECG-gated imaging method includes, e.g., an imaging method in which imaging is performed after a predetermined delay time from a position of an R-wave of an ECG signal, and also includes an imaging method in which imaging is performed in a specific cardiac phase such as diastole or systole. However, patients with heart disease have a problem that the cardiac cycle becomes unstable and imaging cannot be performed at a desired synchronization timing.

As a countermeasure for the above-described problem, an antiarrhythmic effect can be obtained by performing RIC as described above. In other words, by applying ischemia and reperfusion repeatedly to a patient with heart disease, the cardiac cycle can be stabilized and thus imaging at a desired synchronization timing becomes possible. As a result, imaging capability can be improved.

As mentioned above, the blood-flow increasing effect and the antiarrhythmic effect are obtained by the RIC. In addition, RIC has the effect of preventing or reducing a disorder, e.g., IRI (ischemia-reperfusion injury), that may occur with the treatment of a disease such as myocardial infarction or cerebral infarction. In this case, the RIC is classified into three types according to the timing of applying RIC: RI-preC (remote ischemic preconditioning), RI-perC (remote ischemic perconditioning), and RI-postC (remote ischemic postconditioning). Of these, RI-preC is applied before “ischemia” in ischemia-reperfusion injury, RI-perC is applied after the onset of “ischemia” in ischemia-reperfusion injury, and RI-postC is applied during “perfusion” in ischemia reperfusion injury. Note that “ischemia” and reperfusion in “ischemia-reperfusion injury” and ischemia and reperfusion in the “RIC” described so far are different phenomena.

(Detailed Operation)

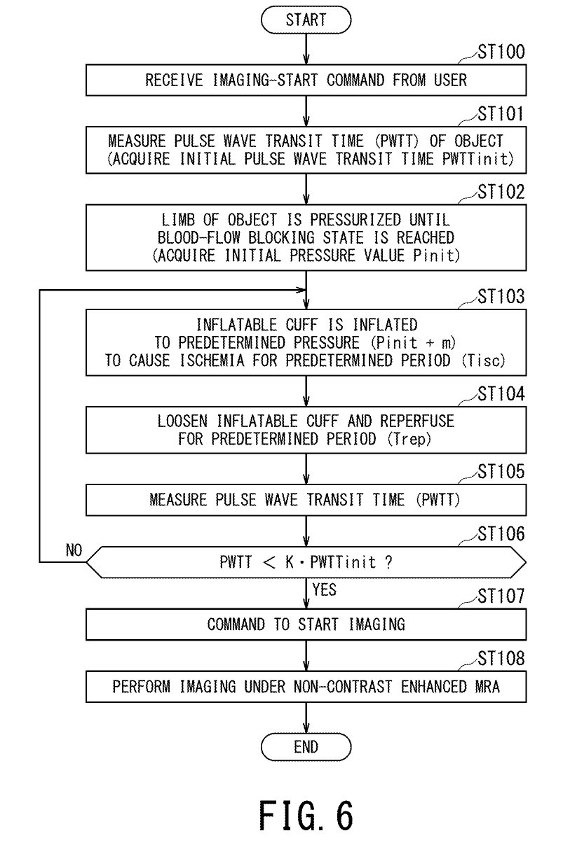

FIG. 6 is a flowchart illustrating a detailed operation of the MRI system 200 and the MRI apparatus 1 of the first embodiment. Note that FIG. 6 is also a flowchart illustrating the procedure of the MRI method of the present embodiment.

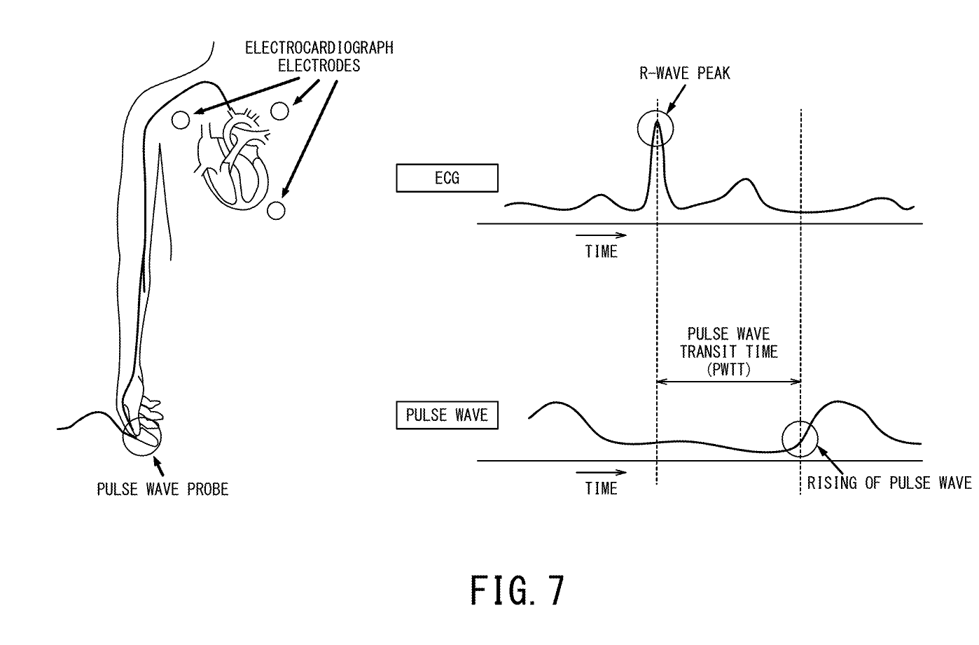

FIG. 7 is a schematic diagram illustrating a concept of a pulse-wave transit time.

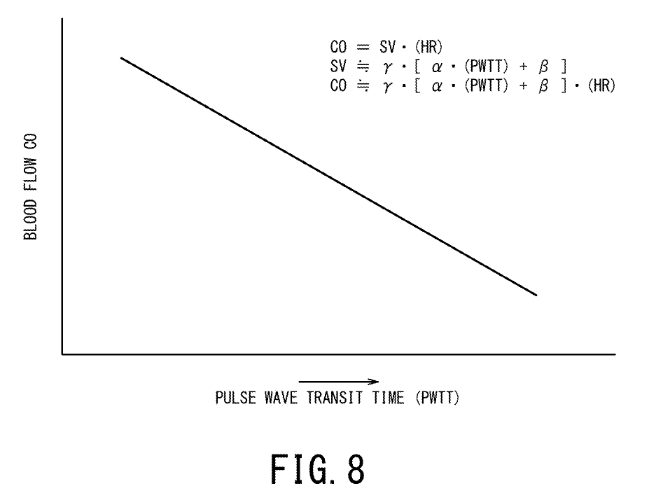

FIG. 8 is a graph illustrating relationship between a pulse-wave transit time and blood flow.

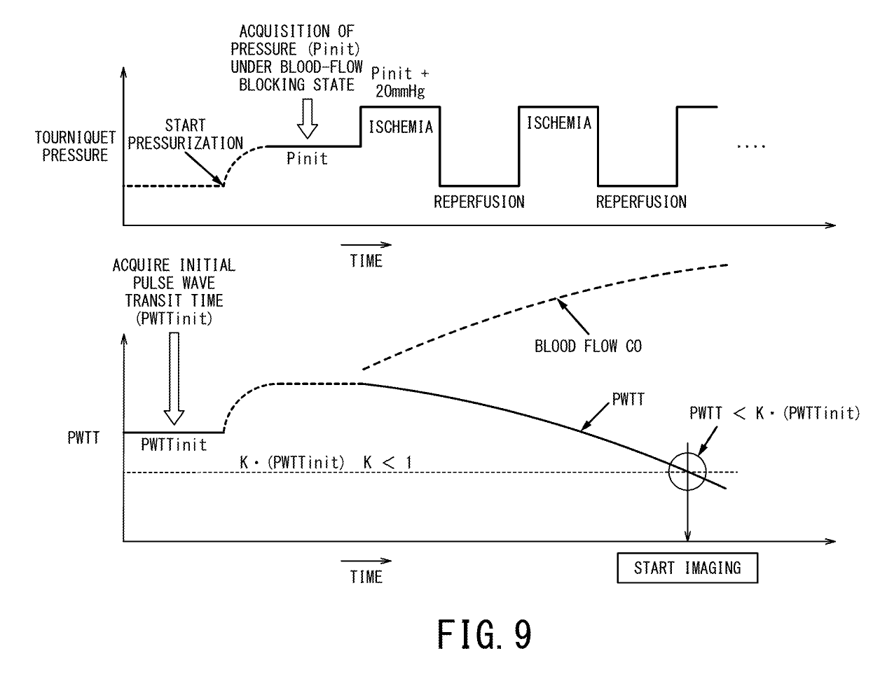

FIG. 9 is a timing chart illustrating the operation concept of the MRI system 200 and the MRI apparatus 1 according to the first embodiment;

On the basis of the step number shown in the flowchart of FIG. 6, the detailed operation of the MRI system 200 and the MRI apparatus 1 according to the first embodiment will be described below by referring to FIG. 7 to FIG. 9 as required.

First, in the step ST100 of FIG. 6, the RIC-combined imaging control function 404 of the processing circuitry 40 receives the imaging start command from a user. The imaging start command is inputted to the RIC-combined imaging control function 404 by the user operating the input interface 43.

The operation performed by the user is only commanding to start imaging via the input interface 43 in the step ST100, and thereafter, every processing until actual imaging is executed (i.e., the processing from the steps ST101 to ST108) is automatically performed by the MRI system 200 and the MRI apparatus 1. Thus, in the following RIC-combined imaging, a further operational burden on the user hardly occurs.

After receiving the imaging start command in the step ST100, in the next step ST101, the pulse wave transit time (PWTT) of the object is measured. The pulse wave transit time measured at this timing is the initial pulse wave transit time (PWTTinit). The pulse wave transit time is measured by the electrocardiograph 201, the pulse wave meter 202, and the PWTT measurement function 401 of the processing circuitry 40.

As shown in FIG. 7, the pulse wave transit time is defined as time difference between the rising time of the pulse wave outputted from the pulse wave meter 202 and the peak time of the R-wave included in the ECG signal that is outputted from the electrocardiograph 201. The probe of the pulse wave meter 202 (e.g., a probe configured to optically measure percutaneous oxygen saturation (SpO₂)) is usually attached to the fingertip of the hand. Thus, the pulse wave transit time can generally be regarded as the time needed for the arterial blood pumped from the heart to propagate to the fingertip of the hand. The PWTT measurement function 401 detects the peak time of the R-wave and the rising time of the pulse wave so as to measure the pulse wave transit time by calculating the time difference between both.

In the present embodiment, the reason for measuring the pulse wave transit time is to evaluate the blood-flow increasing effect caused by RIC. In other words, it is possible to indirectly evaluate the blood flow by measuring the pulse wave transit time as described below.

As shown by the following equation (1), it is known that there is negative correlation between the pulse wave transit time (PWTT) and cardiac stroke volume SV for one heartbeat.

SV≈γ·[α·(PWTT)+β] Equation (1)

In the equation (1), α is a negative constant, and β and γ are positive constants.

The blood flow CO (i.e., the stroke volume per minute) is indicated as the product of the stroke volume SV for one heartbeat and the heart rate HR for one minute as shown by the following equation (2).

CO=SV·(HR) Equation (2)

From the equations (1) and (2), the following relation holds between the blood flow CO and the pulse wave transit time (PWTT).

CO≈γ·[α·(PWTT)+β]·(HR) Equation (3)

Thus, there is negative correlation between the blood flow CO and the pulse wave transit time (PWTT) as shown in FIG. 8. Accordingly, it is possible to indirectly evaluate the blood flow CO by measuring the pulse wave transit time (PWTT). Specifically, the initial value of the blood flow CO at the time of measuring the initial pulse wave transit time (PWTTinit) is defined as COinit. When the pulse wave transit time (PWTT) decreases less than its initial value (PWTTinit), the blood flow CO can be regarded as having increased more than its initial value (COinit).

The measurement of the initial pulse wave transit time (PWTTinit) in the step ST101 is performed before the pressurization performed by the pressure device 203 (see the left side of the timing chart in the lower part of FIG. 9).

Returning to FIG. 6, in the next step ST102, one of the four limbs (e.g., the upper arm) of the object is pressurized until the blood-flow is completely stopped. The pressure at the time of the complete blood-flow stoppage is acquired and held as an initial pressure value Pinit (mmHg).

The processing of the step ST102 is performed in such a manner that the pressure-device control function 403 receives a pressure-start command from the RIC-combined imaging control function 404 of the processing circuitry 40, and then controls the pressure device 203. For instance, the pressure-device control function 403 gradually increases the pressure applied by the inflatable cuff 204 of the pressure device 203 while mentoring the waveform of the peripheral pulse wave (i.e., the pulse wave of the fingertip) obtained from the pulse wave meter 202. Afterward, when the waveform of the peripheral pulse wave becomes almost flat, the pressure-device control function 403 determines that the complete blood-flow stoppage has been reached, and temporarily stops the increase in pressure applied by the inflatable cuff 204 of the pressure device 203. Then, the pressure of the inflatable cuff at the time is acquired as the initial pressure value Pinit (mmHg).

The execution timing of the step ST102 is shown on the left side of the timing chart of the upper part of FIG. 9.

The processing from the steps ST103 to ST106 corresponds to the processing for increasing the blood flow by executing the ischemia-and-reperfusion cycle.

First, in the step ST103, the pressure-device control function 403 causes the pressure device 203 to inflate the inflatable cuff 204 to a predetermined pressure for a predetermined period Tisc to stop the blood flow and cause ischemia condition. Here, the predetermined pressure is the pressure (Pinit+m) obtained by adding a predetermined margin m to the initial pressure value Pinit, and is, e.g., a pressure such as Pinit+20 (mmHg). By applying a pressure larger than the initial pressure value Pinit by the margin, it is possible to more reliably cause the ischemic condition. Additionally, the predetermined period Tisc may be determined by preliminary study such as a clinical experiment, and is, e.g., a period of about 5 minutes.

After applying the pressure for the predetermined period, in the next step ST104, the pressure-device control function 403 causes the pressure device 203 to loosen the inflatable cuff 204 (i.e., leave the pressure unapplied) in order to cause reperfusion. The reperfusion period Trep may also be determined by preliminary study such as a clinical experiment, and is, e.g., a period of about 3 minutes to about 5 minutes.

In the next step ST105, the pulse wave transit time PWTT is measured. The method of measuring the pulse wave transit time PWTT is the same as that of the step ST101. In the step ST105, the pulse wave transit time PWTT is measured during the execution of ischemia-and-reperfusion cycle after the pressurization, whereas the initial pulse wave transit time PWTTinit before the pressurization is measured in the step ST101.

The step ST106 is the processing of determining whether or not to start imaging. The determination of the step ST106 is performed by the imaging-start-timing determination function 402. Specifically, the imaging-start-timing determination function 402 determines the imaging start timing by using the ratio between the initial pulse wave transit time PWTTinit measured in the step ST101 before the pressurization (or before the start of the ischemia-and-reperfusion cycle) and the pulse wave transit time PWTT measured in the step ST105 after the start of the ischemia-and-reperfusion cycle).

For instance, a value obtained by multiplying the initial value PWTTinit by a predetermined constant K (K<l) is compared with the pulse wave transit time PWTT measured in the step ST105, and it is determined whether PWTT is smaller than K·PWTTinit (PWTT<K·PWTTinit) or not.

As described above, there is negative correlation between the blood flow CO and the pulse wave transit time PWTT. Thus, when the pulse wave transit time PWTT is equal to or larger than K·PWTTinit, it is determined that the blood-flow increasing effect by RIC and the antiarrhythmic effect are not sufficiently obtained, and the processing returns to the step ST103 and the ischemia-and-reperfusion cycle is repeated. The ischemia-and-reperfusion cycle is repeated until it is determined in the step ST106 that the pulse wave transit time PWTT becomes smaller than K·PWTTinit.

In the step ST106, when it is determined that the pulse wave transit time PWTT becomes smaller than K-PWTTinit, the processing proceeds to the step ST107.

In the next step ST107, for instance, in response to reception of the affirmative determination result of the imaging-start-timing determination function 402, the RIC-combined imaging control function 404 issues a command for the sequence controller 34 to start imaging.

In the next step ST108, the scanner 600 executes imaging under non-contrast enhanced MRA in response to the imaging start command.

FIG. 9 illustrates the processing of the above-described steps ST101 to ST107 by using a timing chart shown in the upper part for illustrating change in the pressure of the inflatable cuff 204 and another timing chart shown in the lower part for illustrating change in the pulse wave transit time PWTT. As can be seen from FIG. 9, by repeating the ischemia-and-reperfusion cycle, the blood flow gradually increases due to the RIC effects. Further, the increase in blood flow is evaluated by the degree of decrease in pulse wave transit time PWTT and is used for determining the imaging start timing.

According to the MRI system 200, the MRI apparatus 1, and the MRI method of the first embodiment described above, the RIC effects of RIC-combined imaging make it possible to enhance the imaging capability of blood vessels for patients with decreased blood flow due to circulatory failure or patients with unstable cardiac cycles.

Additionally, in the RIC-combined imaging of the first embodiment, a user has only to input a command to start imaging. In other words, as long as the user inputs a command to start imaging, the system or the apparatus automatically starts the processing, and thus, there is no case where any other operational burden is imposed on the user.

Further, in the present embodiment, the imaging start timing is indirectly determined on the basis of the pulse wave transit time with the use of a low-cost device such as a pulse wave meter and an electrocardiograph. Thus, in the present embodiment, it is possible to determine the imaging start timing by using a relatively low-cost device such as a pulse wave meter and an electrocardiograph, without requiring a special device for directly measuring increase in blood flow.

In addition, the MRI system 200 and the MRI apparatus 1 of the first embodiment can be configured to stop the cycle of ischemia and reperfusion by the pressure device 203 so as to stop pressurization by the pressure device 203 by using the biological information such as the measured pulse wave transit time.

Note that, although, in FIG. 1 and FIG. 2, the MRI apparatus 1 itself does not include the electrocardiograph 201, the pulse wave meter 202, or the pressure device 203, embodiments of the present invention are not limited to such configuration. For instance, the MRI apparatus 1 may be configured to include all, arbitrary two, or any one of the electrocardiograph 201, the pulse wave meter 202, and the pressure device 203.

### Second Embodiment

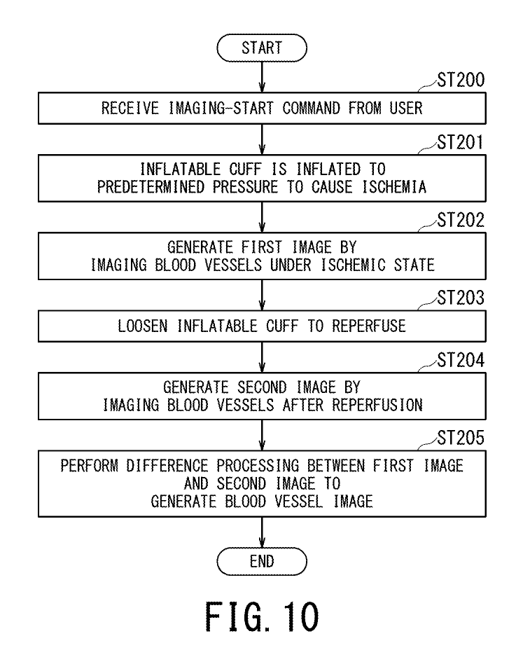

FIG. 10 is a flowchart illustrating an operation of the MRI system 200, the MRI apparatus 1, and the MRI method according to the second embodiment. The configuration of each of the MRI system 200 and the MRI apparatus 1 according to the second embodiment is the same as that shown in FIG. 1.

First, in the step ST200, the processing circuitry 40 receives an imaging start command from a user. The imaging start command is inputted from the input interface 43 to the processing circuitry 40 with the user operation.

In the next step ST201, the processing circuitry 40 causes the pressure device 203 to apply pressure from the inflatable cuff 204 to the object so that the blood vessels to be imaged are brought into the ischemic condition.

Thereafter, in the next step ST202, the processing circuitry 40 causes the entire MRI system 200 to perform the first imaging on the blood vessels in the ischemic condition so as to generate the first image, using a predetermined non-contrast enhanced MRA method (e.g., the FBI method). The start timing of the first imaging is determined on the basis of, e.g., the waveform of the biological information acquired from the pulse wave meter 202 (i.e., pulse wave). For instance, by determining whether the waveform of the pulse wave acquired from the pulse wave meter 202 has become substantially flat or not, the processing circuitry 40 can determine whether the blood vessel has reached the ischemic condition or not.

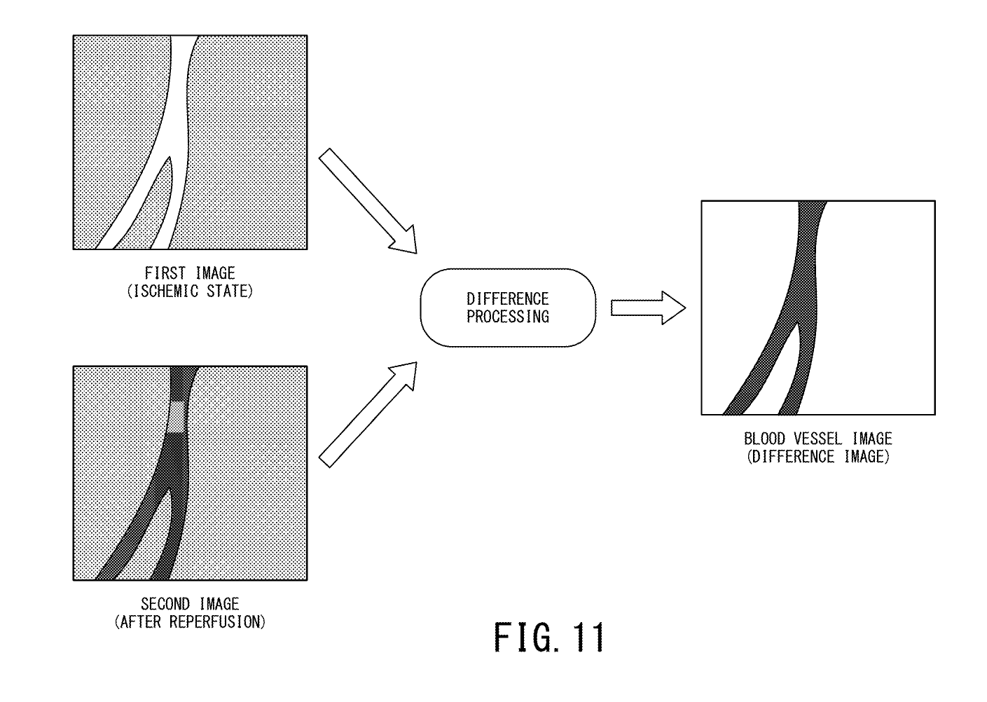

The upper left part of FIG. 11 schematically illustrates the first image that has been imaged in the ischemic condition. The white region in the first image indicates a blood-vessel region in the ischemic condition, and the surrounding gray region indicates the background of the blood vessel(s). In blood vessels in the ischemic condition, the blood flow considerably decreases, and the blood vessels cannot been satisfactorily depicted by many imaging method belonging to non-contrast enhanced MRA. For instance, each pixel value of the blood-vessel region shows a lower value than that of the background.

After completion of the first imaging, in the next step ST203, the processing circuitry 40 causes the pressure device 203 to loosen the inflatable cuff 204 so that blood is reperfused in the target region.

Thereafter, in the next step ST204, the processing circuitry 40 causes the entire MRI system 200 to perform the second imaging on the blood vessels subjected to the reperfusion so as to generate the second image, using the same type of non-contrast enhanced MRA method as the first imaging. The start timing of the second imaging is determined on the basis of the waveform of the biological information acquired from the pulse wave meter 202 (i.e., pulse wave), similarly to the first imaging. For instance, the processing circuitry 40 can determine whether the blood vessels have returned from the ischemic condition to the reperfusion condition or not, by determining whether the waveform of the pulse wave acquired from the pulse wave meter 202 has returned from the flat wave to a standard pulse wave or not, respectively.

The lower left part of FIG. 11 schematically illustrates the second image that has been imaged after reperfusion (i.e., after the blood vessel has been reached to the reperfusion condition). The black region in the second image indicates the blood-vessel region after reperfusion, and the gray region around it indicates the background of the blood vessel(s). Since the blood flow in the blood vessels after reperfusion has returned to a normal condition, those blood vessels can be satisfactorily depicted by many imaging methods belonging to non-contrast enhanced MRA. For instance, each pixel value of the blood-vessel region indicates a higher value than that of the background.

In the next step ST205, the processing circuitry 40 performs difference processing between the first image and the second image so as to generate a blood vessel image.

The right side of FIG. 11 illustrates a difference image between the first image and the second image. By performing the difference processing between the first image and the second image, it is possible to generate a blood vessel image in which the background region is suppressed and only the blood vessel region is depicted.

Note that the blood vessel(s) to be imaged is not necessarily limited to an artery, and a vein can also be an imaging target.

In a conventional FBI method, difference processing is performed between an image acquired in systole and an image acquired in diastolic, each of the images are acquired under electrocardiographic synchronization. However, as mentioned above, in the second embodiment, the imaging timing can be set regardless of cardiac time phase, and thus, there is no need to perform ECG synchronization.

### Third Embodiment

The MRI apparatus 1 and the MRI system 200 according to the third embodiment can acquire images and data useful for diagnosis and treatment of, e.g., cerebral infarction, by performing RIC as described above, i.e., by using the pressure device 203 for causing ischemia and reperfusion with respect to an object.

Cerebral infarction is a disease in which blood vessels in the brain are blocked. When blood vessels in the brain become blocked, blood does not flow beyond the blocked part and deficiency of oxygen and nutrition is caused. When this state continues long, brain cells are necrosed and various obstacles such as limb paralysis and language disorder will occur.

Cerebral infarction in the early stages, i.e., cerebral infarction with a short elapsed time since onset is called acute cerebral infarction. This acute cerebral infarction is the target for the MRI apparatus 1 and the MRI system 200 of the third embodiment.

The aim of treating acute cerebral infarction is to restore blood flow as early as possible, e.g., within hours from the onset. As a main treatment method for acute cerebral infarction, there is a known method of melting a thrombus with a drug such as alteplase (rt-PA), and there is also a known method of removing a thrombus in an artery by using a device such as a stent.

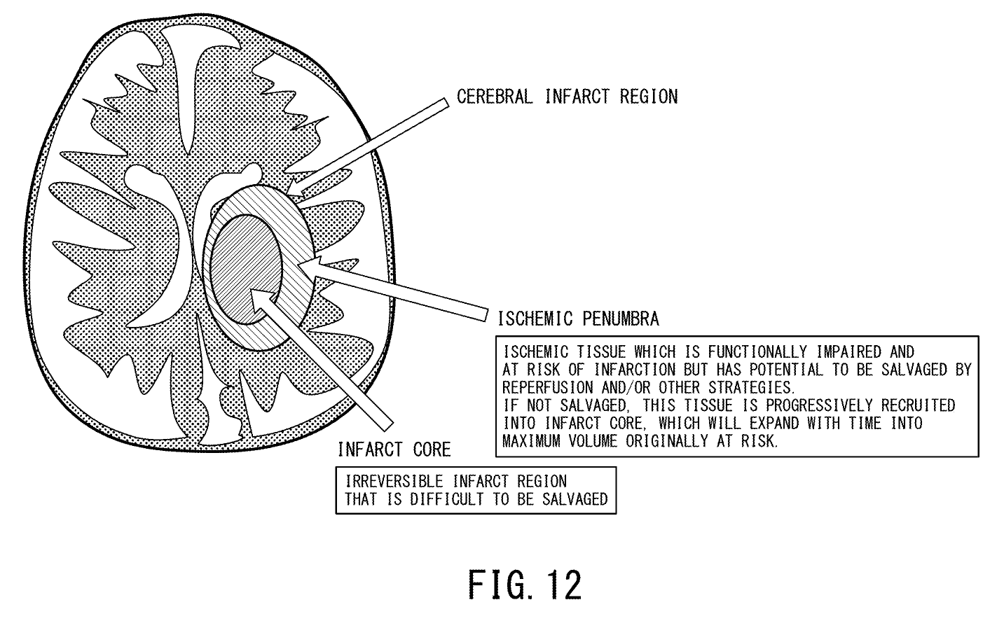

FIG. 12 is a schematic diagram illustrating an axial cross-section of the brain in which acute cerebral infarction occurs. It is said that an acute cerebral infarct region (hereinafter, simply referred to as a cerebral infarction region) is composed of a region called an “infarct core” indicated by the dark ellipse and a region called an “ischemic penumbra” indicated by the obliquely hatched ellipse in FIG. 12.

The infarct core is a region that is undergoing structural destruction such as necrosis due to severe ischemia. The infarct core is an irreversible infarct site and is a region that is difficult to be salvaged.

On the other hand, the ischemic penumbra is a region between the normal region and the infarct core to be positioned around the infarct core, and is a region suffering from moderate ischemia. For instance, the ischemic penumbra is defined as follows: “the ischemic penumbra is a tissue functionally impaired and at the risk of infarction but has the potential to be salvaged by reperfusion and/or other strategies. If not salvaged, this tissue is progressively recruited into a new infarct core, which will expand with time”.

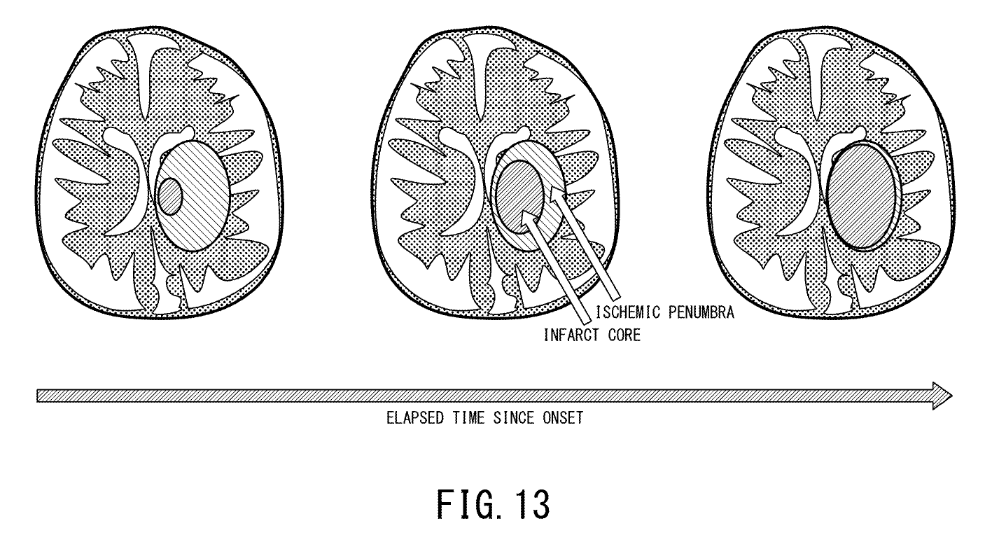

FIG. 13 is a schematic diagram illustrating respective stages of a cerebral infarction region in which a region of an infarct core, which is difficult to be salvaged, gradually increases but a region of an ischemic penumbra, which has the potential to be salvaged, gradually decreases with elapsed time since onset. In the treatment of acute cerebral infarction, super-early treatment is important, and for this purpose, it is important to detect ischemic penumbra, which is a region having the potential to be salvaged (i.e., treatable region), at an early stage.

As a conventional method for detecting an ischemic penumbra, there is a known method (i.e., DWI-PWI mismatch method) that uses a mismatch between DWI (Diffusion Weighted Imaging) and PWI (Perfusion Weighted Imaging). In the DWI-PWI mismatch method, the difference region between the infarct region detected by DWI and the infarct region detected by PWI is determined to be the ischemic penumbra region.

However, as is pointed out, there is a problem that the DWI-PWI mismatch method cannot clearly distinguish the boundary between the infarct core and the ischemic penumbra.

In addition, PWI is an invasive imaging method using a contrast medium. In the treatment of acute cerebral infarction, PWI may be performed every several hours for evaluation before and after treatment. For this reason, PWI is a heavy burden on a patient.

Since PWI is imaging for measuring a temporal change in contrast medium concentration after injection of the contrast medium, imaging in plural time phases is required and information on perfusion is acquired from data of a relatively long imaging time. However, in acute cerebral infarction, the circulation dynamics of blood flow change dynamically during the long imaging time of the PWI. In other words, the imaging target, of which circulatory dynamics change with time, will be imaged during the long imaging time of the PWI, and thus the detection accuracy for the ischemic penumbra is limited.

The third embodiment described below solves the above-described problem and provides the MRI apparatus 1 and the MRI system 200, both of which can distinguish between the infarct core and the ischemic penumbra without performing PWI, and can reliably detect presence/absence of the ischemic penumbra having the potential to be salvaged and its positional range in a short time if present.

The third embodiment is mainly based on two points of view. The first point of view is to use the IVIM (intravoxel incoherent motion) method as the imaging method of the MRI apparatus 1. The second point of view is to combine the imaging with RIC (remote ischemic conditioning) using the pressure device in a manner similar to the first and second embodiments. Since the RIC in the third embodiment is performed after the onset of ischemia associated with cerebral infarction, it corresponds to the RI-perC (remote ischemic perconditioning) out of the above three types of RIC.

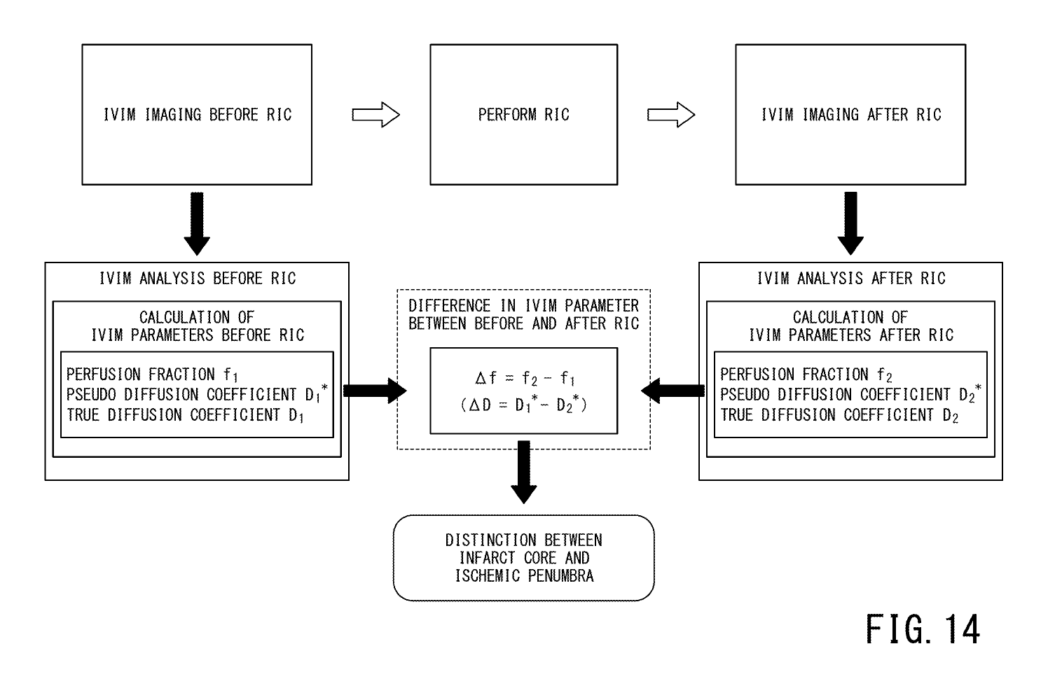

FIG. 14 is a schematic diagram illustrating the processing concept of the third embodiment. As shown in FIG. 14, in the third embodiment, the IVIM imaging is performed before RIC, as the first IVIM imaging, then RIC is performed, and then the IVIM imaging is performed again after the RIC, as the second IVIM imaging.

As described below, the IVIM imaging is diffusion weighted imaging that uses plural b-values. In the IVIM imaging, IVIM parameters such as a true diffusion coefficient D, a pseudo diffusion coefficient D*, and a perfusion fraction f are calculated for each voxel from data acquired by plural DWI sequences, b-values of which are set to values different from each other. In this specification, the term “voxel” is assumed to include a pixel in a two-dimensional image. Each of the IVIM parameters will be described below in detail.

In the third embodiment, as shown in FIG. 14, the true diffusion coefficient D₁, the pseudo diffusion coefficient D₁*, and the perfusion fraction f₁ are calculated for each voxel from data acquired by the first IVIM imaging before RIC. Similarly, the true diffusion coefficient D₂, the pseudo diffusion coefficient D₂*, and the perfusion fraction f₂ are calculated for each voxel from the data acquired by the second IVIM imaging after RIC. Further, the difference between IVIM parameters before and after RIC is calculated. Specifically, one of the difference Δf (=f₂−f₁) in perfusion fraction before and after RIC and the difference ΔD* (=D₂*−D₁*) in pseudo diffusion coefficient is calculated.

Typically, the difference Δf in perfusion fraction before and after RIC is calculated for each voxel.

Further, the processing circuitry 40 distinguishes between the infarct core and the ischemic penumbra by determining whether each target voxel is a voxel in the infarct core or a voxel in the ischemic penumbra, on the basis of magnitude of the difference Δf in perfusion fraction, magnitude of the difference ΔD* in pseudo diffusion coefficient, or the combination of both.

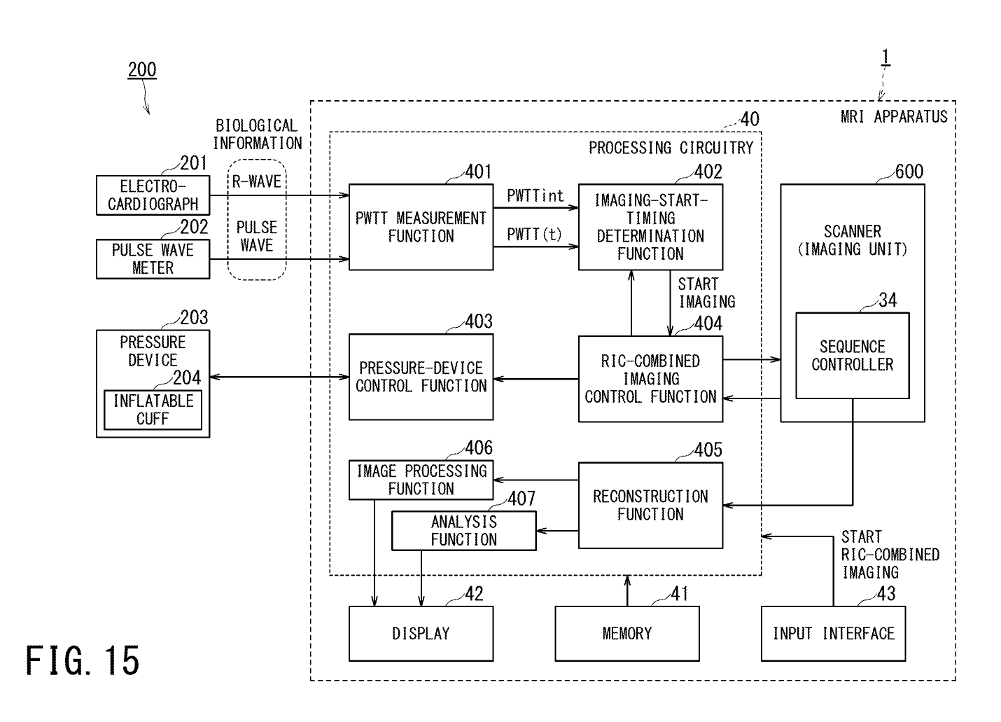

FIG. 15 is a block diagram illustrating configuration related to the RIC-combined imaging of the MRI system 200 and the MRI apparatus 1 according to the third embodiment. The MRI system 200 (MRI apparatus 1) of the third embodiment includes an analysis function 407 for analyzing data of the reconstructed image that is generated by the reconstruction function 405. In the third embodiment, the RIC-combined imaging control function 404 controls the scanner 600 such that the IVIM imaging is performed. Since other configuration and functions of the third embodiment are the same as those of the first embodiment (FIG. 2), duplicate description is omitted.

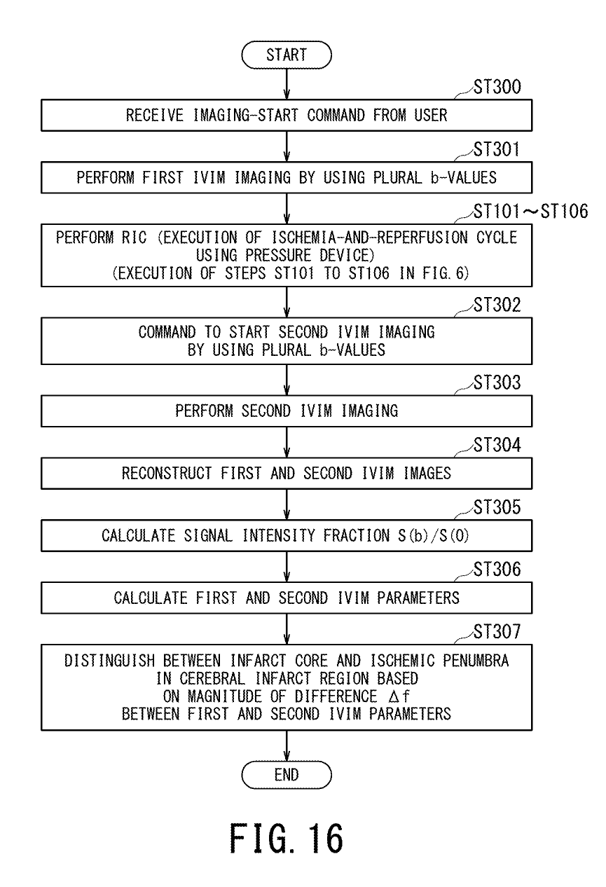

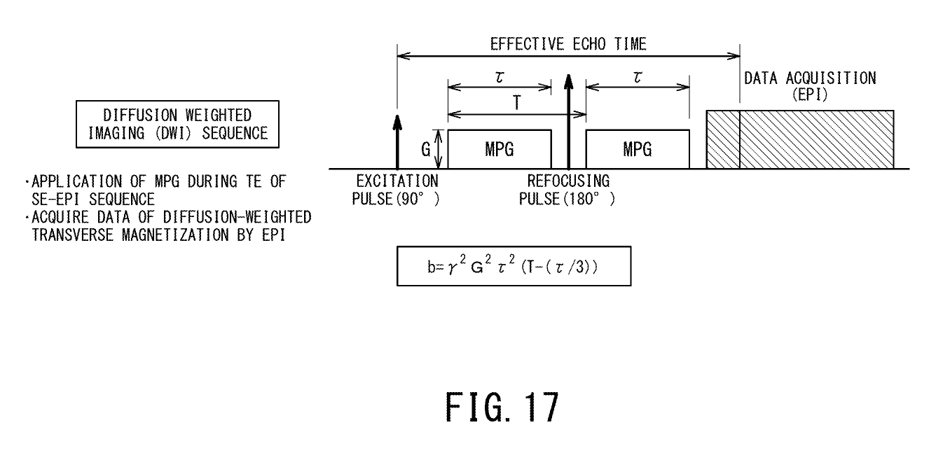

FIG. 16 is a flowchart illustrating an operation of the MRI system 200 and the MRI apparatus 1 according to the third embodiment. On the basis of the step number shown in the flowchart of FIG. 16, a description will be given of the analysis function 407 and the RIC-combined imaging control function 404 in the third embodiment by referring to FIG. 17 to FIG. 20 as required.

First, in the step ST300, the RIC-combined imaging control function 404 receives the imaging start command from a user via the input interface 43.

In response to the reception of the imaging start command, in the next step ST301, the RIC-combined imaging control function 404 causes the scanner 600 to perform the first IVIM imaging with the use of plural b-values (i.e., the IVIM imaging before RIC).

FIG. 17 is a schematic diagram illustrating a pulse sequence for the IVIM imaging. The pulse sequence of the IVIM imaging is the same as the pulse sequence of DWI (diffusion weighted imaging). For instance, as shown in FIG. 17, the pulse sequence of the IVIM imaging is based on a pulse sequence of SE(spin echo)-EPI(echo planer imaging) and further includes a pair of gradient pulses called MPG (motion probing gradient) pulses that are applied before and after the refocusing pulse.

The period indicated by the obliquely hatched rectangle subsequent to the second MPG pulse is a data acquisition period under EPI. In single-shot SE-EPI, data of all the phase encodes corresponding to one slice are acquired during this data acquisition period. By applying the pulse sequence shown in FIG. 17 to different slices plural times, it is possible to acquire desired three-dimensional volume data that corresponds to one b-value. In the case of two-dimensional data acquisition (i.e., in the case of acquiring data of one slice), the data of one slice corresponding to one b-value can be acquired by performing the pulse sequence shown in FIG. 17 only once.

In the IVIM imaging, this data acquisition is performed plural times, in each of which the b-value is changed, and plural data sets corresponding to respective different b-values are acquired. The b-value is indicated by the following equation (4) as also shown in FIG. 17.

b=γ²·G²·τ²[T−(τ/3)] Equation (4)

In the equation (4), γ is the magnetogyric ratio, G is magnitude of the gradient field of each MPG pulse, τ is pulse length of each MPG pulse, and T is the spacing between the respective leading edges of the two MPG pulses. The b-value can be changed by changing at least one value of G, τ, and T. For instance, by setting the pulse length τ of each MPG pulse to different values, the b-value can be changed.

In the step ST301, the RIC-combined imaging control function 404 causes the scanner 600 to perform the first IVIM imaging before RIC on the basis of the pulse sequence of DWI having different b-values as described above.

Next, RIC is performed. This RIC is the same as the processing from the steps ST101 to ST106 of the first embodiment. Specifically, the ischemia-and-reperfusion cycle is repeated once or plural times on the arm of the object by using the inflatable cuff 204 of the pressure device 203. By performing RIC, the blood-flow increasing effect is obtained as one of the RIC effects.

In the step ST106, it is determined whether the blood flow has increased to a desired amount or not, on the basis of the pulse wave transit time PWTT. When it is determined that the blood flow has increased to the desired amount, the processing proceeds to the step ST302.

In the step ST302, the imaging-start-timing determination function 402 issues a command for the RIC-combined imaging control function 404 to start the second IVIM imaging.

In the next step ST303, the RIC-combined imaging control function 404 controls the scanner 600 such that the second IVIM imaging (i.e., IVIM imaging after RIC) is performed. The processing of the second IVIM imaging is exactly the same as the first IVIM, and the only difference lies in that the first IVIM imaging is performed before RIC and the second IVIM imaging is performed after completion of RIC.

In the next step ST304, data acquired in the first IVIM imaging and the second IVIM imaging are reconstructed to generate the first IVIM images and the second IVIM images. The first IVIM images are plural IVIM images corresponding to respective plural b-values, and the same holds true for the second IVIM images. Note that the b-value is assumed to include zero (b=0).

In the next step ST305, the analysis function 407 (FIG. 15) calculates the ratio between the signal intensity S(0) of the image corresponding to one b-value (e.g., b=0) and the signal intensity S(b) of the image corresponding to each of all the b-values, including the b-value of zero (b=0), for each voxel of each of the first IVIM images and the second IVIM images. In other words, the analysis function 407 calculates the ratios including S(0)/S(0), S(1)/S(0), S(2)/S(0), S(n)/S0 for each voxel.

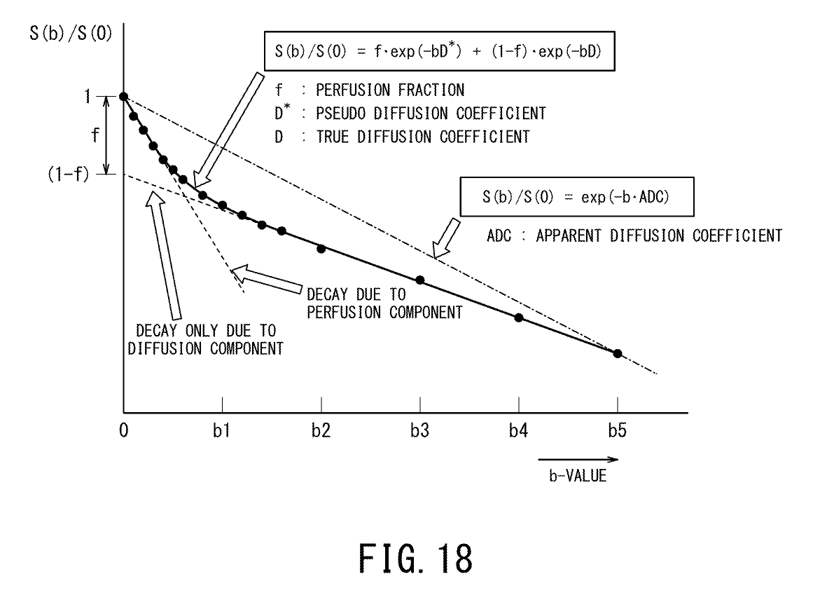

FIG. 18 is a graph illustrating the concept of the signal intensity ratio in a specific one voxel of an IVIM image. In FIG. 18, the horizontal axis indicates the b-value, and the vertical axis indicates the signal intensity ratio S(b)/S(0).

In ordinary DWI, the ratio of the signal intensity S(b) to the signal intensity S(0) (i.e., when b=0) is supposed to decay according to a single exponential model of the following equation (5).

S(b)/S(0)=exp(−b·ADC) Equation (5)

In the equation (5), ADC is called an apparent diffusion coefficient. In other words, it is assumed that an MR signal in ordinary DWI decays in accordance with the diffusion component represented by the parameter ADC due to application of the MPG pulses. The one-dot chain line in FIG. 18 represents the equation (5) when S(b)/S(0) is logarithmically plotted.

On the other hand, in the IVIM imaging, the application of the MPG pulses is assumed to cause each voxel to include two components, in which one of the components decays in accordance with diffusion based on random motion of water molecules, and another component decays in accordance with incoherent microcirculation in the capillary (i.e., a component decaying in accordance with perfusion). Specifically, in the IVIM imaging, it is assumed that the ratio of the signal intensity S(b) to the signal intensity S(0) decays in accordance with a bi-exponential model shown by the following equation (6).

S(b)/S(0)=f·exp(−b·D*)+(1−f)·exp(−b·D) Equation (6)

In the equation (6), the first term on the right side is a term corresponding to decay due to perfusion, and D* is called a pseudo diffusion coefficient. The second term on the right side is a term corresponding to decay due to diffusion, and D is called a true diffusion coefficient. Further, f represents the ratio of decay due to perfusion with respect to the entire decay, and is called a perfusion fraction.

The black dots in FIG. 18 are logarithmic plots obtained by acquiring the signal intensity ratio S(b)/S(0) from the data acquired in the IVIM imaging and logarithmically plotting them for each b-value. From the plurality of actually measured values represented by the black dots and the bi-exponential model shown by the equation (6), it is possible to calculate the IVIM parameters including the pseudo diffusion coefficient D*, the true diffusion coefficient D, and the perfusion fraction f by using a known method such as curve fitting.

These IVIM parameters are calculated for each voxel. Thus, by placing each parameter value at the position of each voxel, it also may be possible to generate a pseudo diffusion coefficient D map, a true diffusion coefficient D map, and a perfusion fraction f map.

As can be understood from FIG. 18, in the region where the b-value is small, steep decay is shown with respect to the b-value, and this steep decay is dominated by the decay due to perfusion (i.e., decay due to the pseudo diffusion coefficient D*). Contrastively, in the region where the b-value is large, gentle decay is shown with respect to the b-value, and this gentle decay is dominated by the decay due to diffusion (i.e., decay due to the true diffusion coefficient D).

Returning to FIG. 16, in the step ST306, the first and second IVIM parameters are calculated by the above-described method. In other words, the pseudo diffusion coefficient D₀*, the true diffusion coefficient D₀, and the perfusion fraction f₀ are calculated for each voxel as the first IVIM parameters of the IVIM imaging performed before RIC. Similarly, the pseudo diffusion coefficient Dₐ*, the true diffusion coefficient Dₐ, and the perfusion fraction fₐ are calculated for each voxel as the second IVIM parameters of the IVIM imaging performed after RIC.

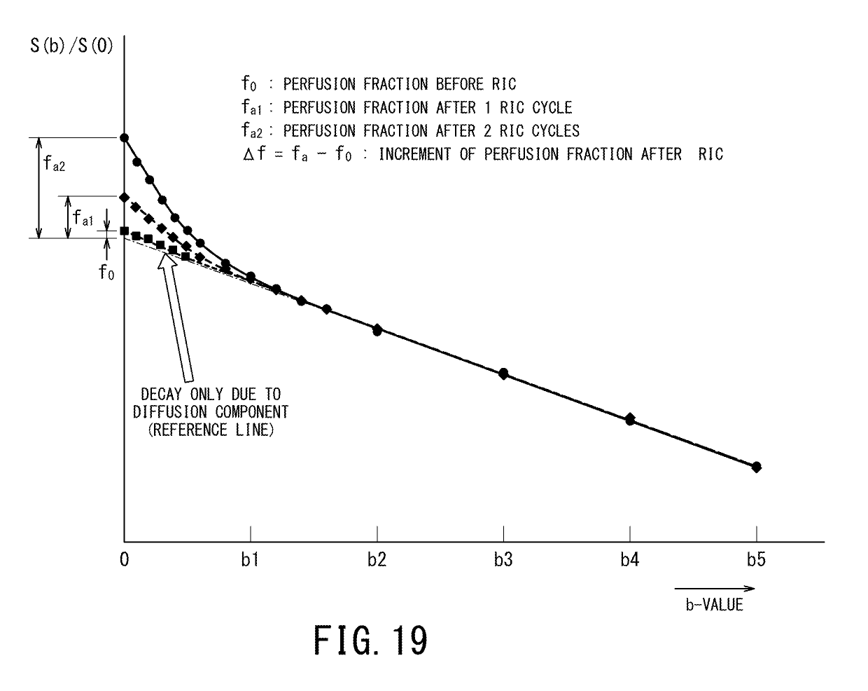

FIG. 19 is a graph schematically illustrating change in characteristics of signal intensity ratio with respect to the b-value in a voxel of an ischemic penumbra region before and after RIC. In FIG. 19, the curve connecting black square dots indicates the signal-intensity-ratio curve before RIC, the curve connecting the black rhombus dots indicates the signal-intensity-ratio curve after performing the RIC cycle once, and the curve connecting black circle dots indicates the signal-intensity-ratio curve after performing the RIC cycle twice.

In acute cerebral infarction, it is known that the microperfusion of the capillary in the cerebral infarct region including the ischemic penumbra decreases, resulting in that the perfusion component decreases as well. Thus, when the IVIM imaging is performed on the brain of a patient suffering from acute cerebral infarction, the perfusion fraction f in the cerebral infarct region shows considerably smaller value than the perfusion fraction f in the normal region of the brain. This is indicated by the signal-intensity-ratio curve (i.e., curve connecting the black square dots) before RIC in FIG. 19. The signal-intensity-ratio curve before RIC is almost superimposed on the decay straight line (i.e., reference line) due only to the diffusion component, and the perfusion fraction f₀ before RIC becomes a small value.

As described in the first embodiment, particularly as described in FIG. 4, blood flow of an object can be increased by performing RIC (i.e., by using the pressure device 203 to cause ischemia and reperfusion for the object). The blood-flow increasing effect by RIC extends to the blood flow of the brain. Thus, RIC increases cerebral blood flow (CBF) and perfusion (i.e., microcirculation of brain capillaries). As a result, the perfusion fraction fₐ after RIC is considered to be larger than the perfusion fraction f₀ before RIC.

For instance, it is considered that the perfusion fraction fₐ₁ after performing the RIC cycle (i.e., the cycle of ischemia and reperfusion) once is larger than the perfusion fraction f₀ before RIC, and the perfusion fraction fₐ₂ after performing the RIC cycle twice becomes further larger than the perfusion fraction fₐ₁.

From the above, the present inventors have worked out an idea that degree of ischemia in a cerebral infarct region can be estimated by increment Δf in perfusion fraction caused by RIC, wherein difference in perfusion fraction between before and after RIC (i.e., increment in perfusion fraction due to RIC) is defined as Δf (=fₐ−f₀).

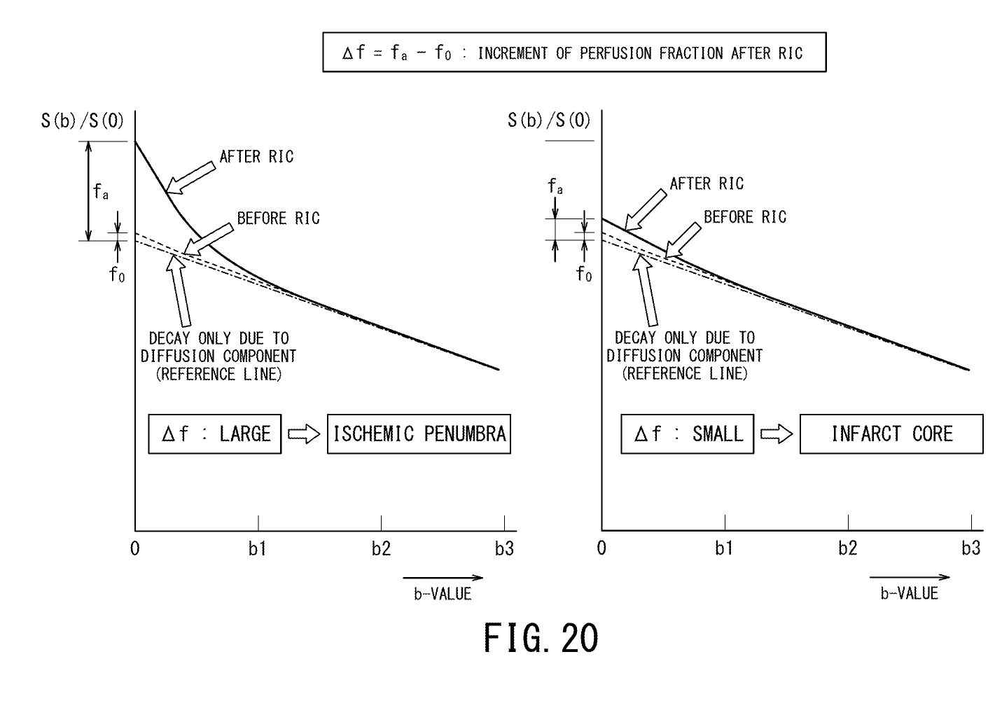

FIG. 20 is a diagram illustrating the concept of the above-described idea. As described above, the ischemic penumbra is a functionally impaired tissue that is at the risk of infarction and subjected to ischemia, but also is a region having the potential to be salvaged by reperfusion and/or other strategies. Thus, as shown in the left graph of FIG. 20, in the ischemic penumbra, the blood flow increases due to execution of RIC, and the increment Δf of the perfusion fraction before RIC is considered to be a relatively large value.

By contrast, the infarct core is an irreversible infarct site and is a tissue that is difficult to be salvaged. Thus, in the infarct core as shown in the right graph of FIG. 20, even when RIC is performed, the blood flow hardly increases and the increment Δf of the perfusion fraction before RIC is considered to be small.

Hence, it is possible to distinguish whether the tissue is an ischemic penumbra or an infarct core on the basis of the magnitude of the increment Δf of the perfusion fraction before and after RIC.

Returning to FIG. 16, in the step ST307, the analysis function 407 distinguishes between the infarct core and the ischemic penumbra in the cerebral infarct region on the basis of the magnitude of the difference Δf between the first IVIM parameter (e.g., the perfusion fraction f₀ acquired in the IVIM imaging performed before RIC) and the second IVIM parameter (e.g., the perfusion fraction fₐ acquired in the IVIM imaging performed after RIC).

For instance, threshold determination is performed for each voxel on the magnitude of the difference Δf in perfusion fraction between before and after RIC, and each voxel larger than the threshold value is determined as a voxel belonging to the ischemic penumbra, while each voxel equal to or below the threshold value is determined as a voxel belonging to the infarct core.

The presence/absence and magnitude of the perfusion component influence not only the perfusion fraction f but also the pseudo diffusion coefficient D*. Thus, instead of the difference Δf in perfusion fraction between before and after RIC, the difference ΔD* in pseudo-diffusion coefficients D* between before and after RIC can be used for distinguishing between the ischemic penumbra and the infarct core. Additionally or alternatively, both the difference ΔD* in pseudo-diffusion coefficient D* and the difference Δf in perfusion fraction may be used for distinguishing between the ischemic penumbra and the infarct core.

According to the MRI apparatus 1 and the MRI system 200 in the third embodiment as described above, it is possible to distinguish between the ischemic penumbra and the infarct core only by performing the non-contrast IVIM imaging without performing contrast-enhanced PWI, and thus any burden is not imposed on a patient. Although there is uncertainty in the distinction between the ischemic penumbra and the infarct core in the PWI-DWI mismatch method, such uncertainty is not included in the third embodiment. Hence, in the third embodiment, it is possible to reliably detect the ischemic penumbra. Further, the MRI apparatus 1 and the MRI system 200 in the third embodiment do not use PWI that requires relatively long imaging time, and thus can determine presence/absence of ischemic penumbra, which is important for the early treatment of acute cerebral infarction, in a shorter time than conventional technology. Similarly, it is possible to detect the region of an ischemic penumbra in a shorter time than conventional technology.

In the third embodiment as mentioned above, the ischemic penumbra and the infarct core in the cerebral infarct region can be distinguished. However, in addition to the above, the RIC-combined imaging can provide various information and various functions useful for image diagnosis.

For example, it is possible to evaluate a change in the biological phenomenon of the object and discriminate between the abnormal region and the normal region of the tissue property in the region of interest of the object, by using results of the first IVIM analysis and the second IVIM analysis. Here, the first IVIM analysis is conducted using data acquired by the first IVIM imaging before the RIC, and the second IVIM analysis is conducted using data acquired by the second IVIM imaging after the RIC.

Alternatively or additionally, the first imaging before the RIC may be performed by the scanner 600 for measuring a biological phenomenon of the object such as BOLD (blood oxygenation level dependent), OEF (oxygen extraction fraction), MRS (magnetic resonance spectroscopy), chemical shift, magnetization transfer, and/or CEST (chemical exchange saturation transfer), and the first data with respect to the above biological phenomenon is acquired through the first imaging. Then, after the execution of the RIC, the second imaging, which has the same imaging method as the first imaging, may be performed to acquire the second data corresponding to the first data.

After that, the first analysis is conducted on the first data to obtain the first analysis result, while the second analysis is conducted on the second data to obtain the second analysis result, by the processing circuitry 40. Then, the change of the biological phenomenon of the object may be evaluated using a result of the first analysis and a result of the second analysis, by the processing circuitry 40. With such evaluation, among the region of interest of the object, the abnormal region and the normal region of the tissue property can be distinguished.

While certain embodiments have been described, these embodiments have been presented by way of example only, and are not intended to limit the scope of the inventions. Indeed, the novel methods and systems described herein may be embodied in a variety of other forms; furthermore, various omissions, substitutions and changes in the form of the methods and systems described herein may be made without departing from the spirit of the inventions. The accompanying claims and their equivalents are intended to cover such forms or modifications as would fall within the scope and spirit of the inventions.

What is claimed is:

1. An MRI apparatus configured to be connected with a pressure device that externally pressures a blood vessel of an object, the MRI apparatus comprising:
a scanner configured to perform imaging on the object; and
processing circuitry configured to
control the pressure device in such a manner that ischemia and reperfusion are caused with respect to the object,
determine a start timing of the imaging based on a state of pressurization caused by the pressure device,
cause the scanner to perform the imaging on the object in accordance with the start timing, and
generate an image by using data acquired in the imaging.

2. The MRI apparatus according to claim 1,
wherein the processing circuitry is configured to determine the start timing based on biological information of the object obtained in response to pressurization of the blood vessel with the pressure device.

3. The MRI apparatus according to claim 1,
wherein the scanner is configured to image the blood vessel by non-contrast enhanced MRA (Magnetic Resonance Angiography) or contrast-enhanced MRA.

4. The MRI apparatus according to claim 1,
wherein the processing circuitry is configured to determine the start timing based on a pulse wave transit time of the object.

5. The MRI apparatus according to claim 1,
wherein the processing circuitry is configured to determine the start timing by using a ratio between a pulse wave transit time measured before pressurization of the blood vessel and a pulse wave transit time measured after causing the ischemia and the reperfusion.

6. The MRI apparatus according to claim 1,
wherein the processing circuitry is configured to control the pressure device in such a manner that a cycle of the ischemia and the reperfusion is repeated.

7. The MRI apparatus according to claim 1,
wherein the processing circuitry is configured to
acquire an initial pressure value by causing the pressure device to pressurize the blood vessel to such an extent that a shape of a peripheral pulse wave becomes flat, before the ischemia and the reperfusion are caused, and
bring the blood vessel into the ischemia by causing the pressure device to pressurize the blood vessel at a pressure that is larger than the initial pressure value by a predetermined margin.

8. The MRI apparatus according to claim 6,
wherein the processing circuitry is configured to
measure biological information derived from blood flow of the object, and
cause the pressure device to stop pressurizing the blood vessel, using the measured biological information.

9. The MRI apparatus according to claim 1,
wherein the processing circuitry is configured to generate a blood vessel image by performing difference processing between a first image acquired in the ischemia and a second image acquired after the reperfusion.

10. An MRI system comprising:
a pressure device configured to externally pressure a blood vessel of an object;
a scanner configured to perform imaging on the object; and
processing circuitry configured to
control the pressure device in such a manner that ischemia and reperfusion are caused with respect to the object,
determine a start timing of the imaging based on a state of pressurization caused by the pressure device,
cause the scanner to perform the imaging on the object in accordance with the start timing, and
generate an image by using data acquired in the imaging.

11. The MRI system according to claim 10,
wherein the pressure device includes a inflatable cuff, and acquires at least one of a blood-flow increasing effect of the object and an antiarrhythmic effect by performing an ischemia-and-reperfusion cycle, in which inflatable cuff pressure is increased to bring the blood vessel into the ischemia and then the blood vessel is brought into the reperfusion by loosening the inflatable cuff.

12. An MRI method comprising:
pressuring a blood vessel of an object to cause ischemia and reperfusion in the object;
determine a start timing of imaging based on a state of pressurization of the blood vessel;
imaging the object in accordance with the start timing; and
generating an image by using data acquired in the imaging.

13. The MRI apparatus according to claim 1, wherein
the scanner is configured to
perform a first imaging for measuring a biological phenomenon of the object to acquire first data, before performing remote ischemic conditioning that includes at least one cycle of the ischemia and the reperfusion, and
perform a second imaging for measuring the biological phenomenon to acquire second data, after performing the remote ischemic conditioning, and
the processing circuitry is configured to
conduct a first analysis on the first data,
conduct a second analysis on the second data, and
evaluate a change of the biological phenomenon of the object, using a result of the first analysis and a result of the second analysis.

14. The MRI apparatus according to claim 1, wherein
the scanner is configured to
perform a first imaging for measuring at least one biological phenomenon of the object including BOLD (blood oxygenation level dependent), OEF (oxygen extraction fraction), chemical shift, MRS (magnetic resonance spectroscopy), magnetization transfer, and CEST (chemical exchange saturation transfer) to acquire first data, before performing remote ischemic conditioning that includes at least one cycle of the ischemia and the reperfusion, and
perform a second imaging for measuring the biological phenomenon to acquire second data, after performing the remote ischemic conditioning, and
the processing circuitry is configured to
conduct a first analysis on the first data,
conduct a second analysis on the second data, and
evaluate a change of the biological phenomenon of the object, using a result of the first analysis and a result of the second analysis.

15. The MRI apparatus according to claim 13, wherein
the scanner is configured to
perform first diffusion weighted imaging by using a plurality of b-values to acquire first data, and
perform second diffusion weighted imaging by using the plurality of b-values to acquire second data, and
the processing circuitry is configured to
conduct a first IVIM (Intravoxel Incoherent Motion) analysis on the first data,
conduct a second IVIM analysis on the first data, and
evaluate a change of the biological phenomenon of the object, using a result of the first IVIM analysis and a result of the IVIM second analysis.

16. The MRI apparatus according to claim 14, wherein
the processing circuitry is configured to evaluate the change of the biological phenomenon of the object to distinguish an abnormal region and a normal region of tissue property among a region of interest of the object.

17. The MRI apparatus according to claim 15, wherein
the processing circuitry is configured to evaluate the change of the biological phenomenon of the object to distinguish an abnormal region and a normal region of tissue property among a region of interest of the object.

18. The MRI apparatus according to claim 15, wherein
the processing circuitry is configured to distinguish between an infarction core and an penumbra in a cerebral infarct region of the object, based on the result of the first IVIM analysis and the result of the second IVIM analysis,
wherein the infarction core is difficult to be salvaged, while the penumbra is a functionally impaired region that has a potential to be salvaged.

19. The MRI apparatus according to claim 18, wherein
the processing circuitry is configured to distinguish between the infarction core and the penumbra by using a first parameter before execution of the remote ischemic conditioning and a second parameter after execution of the remote ischemic conditioning, the first parameter being calculated by the first IVIM analysis, the second parameter being calculated by the second IVIM analysis.

20. The MRI apparatus according to claim 19, wherein
the first parameter is a first perfusion fraction indicating a ratio of perfusion and diffusion before execution of the remote ischemic conditioning;
the second parameter is a second perfusion fraction indicating a ratio of perfusion and diffusion after execution of the remote ischemic conditioning; and
the processing circuitry is configured to distinguish between the infarct core and the penumbra by using the first perfusion fraction and the second perfusion fraction.

21. The MRI apparatus according to claim 20, wherein
the processing circuitry is configured to
calculate a fraction difference between the first perfusion fraction and the second perfusion fraction for each voxel,
determine that each voxel for which the fraction difference is larger than a predetermined first threshold is included in the penumbra, and
determine that each voxel for which the fraction difference is smaller than a predetermined second threshold is included in the infarction core.

22. The MRI apparatus according to claim 19, wherein
the first parameter is a first pseudo diffusion coefficient indicating a degree of signal reduction caused by perfusion before execution of the remote ischemic conditioning;
the second parameter is a second pseudo diffusion coefficient indicating a degree of signal reduction caused by perfusion after execution of the remote ischemic conditioning; and
the processing circuitry is configured to distinguish between the infarct core and the penumbra by using the first pseudo diffusion coefficient and the second pseudo diffusion coefficient.
